(19) United States
(12) Patent Application Publication
(10) Pub. No.: US 2015/0192563 A1
(43) Pub. Date: Jul. 9, 2015

(54) PHASE-DIFFERENCE DETERMINATION USING TEST METER

(71) Applicant: LifeScan Scotland Limited, Inverness (GB)

(72) Inventors: David ELDER, Inverness (GB); Timothy LLOYD, Inverness (GB); Christian FORLANI, Milano (IT); Rossano MASSARI, Milano (IT); Emanuele POZZI, Milano (IT)

(21) Appl. No.: 14/660,001

(22) Filed: Mar. 17, 2015

Related U.S. Application Data
(62) Division of application No. 14/023,275, filed on Sep. 10, 2013.

(51) Int. Cl. G01N 33/49

(52) U.S. Cl. CPC G01N 33/49

(57) ABSTRACT

A hand-held test meter includes a strip port connector to receive a test strip. A signal-measurement circuit applies a periodic voltage signal across a sample applied to the strip and detects a resulting current signal. The circuit provides data of the current signal at a digitizing frequency and a selected phase with respect to the voltage signal. A processor records one or more value(s) of the data and then alters the selected phase. Value(s) are thus recorded at each of a plurality of phases. The processor determines a phase difference of the current signal with respect to the voltage signal using the respective sets of value(s). A method for employing a test meter and a test strip is also disclosed, and includes measuring a respective plurality of points for each of a plurality of different measurement phases and determining a phase difference of a fluid sample therefrom.

### CROSS-REFERENCE

This DIVISIONAL application claims the benefits of priority under 35 USC §§120 and 121 from prior filed U.S. application Ser. No. 14/023,275 filed on Sep. 10, 2013, pending, in which prior filed application is incorporated by reference in its entirety into this application.

### TECHNICAL FIELD

The present invention relates, in general, to medical devices and more specifically to test meters and related methods.

### DESCRIPTION OF RELATED ART

The determination (e.g., detection or concentration measurement) of an analyte in a fluid sample is of particular interest in the medical field. For example, it can be desirable to determine glucose, ketone bodies, cholesterol, lipoproteins, triglycerides, acetaminophen or HbA1c concentrations in a sample of a bodily fluid such as urine, blood, plasma or interstitial fluid. Such determinations can be achieved using a hand-held test meter in combination with analytical test strips (e.g., electrochemical-based analytical test strips).

Hand-held and other portable test meters, e.g., for measuring blood glucose, are intended to be used repeatedly throughout the day. It is therefore desirable that such meters be small and lightweight so that the user will not feel burdened while carrying one. Hand-held test meters generally operate on battery power for portability, so it is also desirable that such meters have a long battery life. It is therefore desirable that analyte-detection circuitry in hand-held test meters be small and lightweight and consume as little energy as possible.

### BRIEF DESCRIPTION OF THE DRAWINGS

Various novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings, in which like numerals indicate like elements, of which:

FIG. 1 is a simplified depiction of a hand-held test meter according to various embodiments;

FIG. 2 is a simplified block diagram of various blocks of the hand-held test meter of FIG. 1 and related components;

FIG. 3 is a graph showing a simulated example of phase adjustment while sampling a current signal according to various exemplary embodiments;

FIGS. 4A and 4B show a simulated example of measuring data used for determining phase differences according to various exemplary embodiments according to a technique referred to herein as “in-sequence undersampling”;

FIGS. 5A and 5B show a simulated example of a technique referred to herein as “out-of-sequence undersampling”;

FIGS. 6A and 6B show a simulated example of a technique referred to herein as “in-sequence phase shifting”;

FIGS. 7A-7C show a simulated example of waveforms and their Fourier transforms;

FIG. 8 shows exemplary circuits that can be used in various embodiments;

FIG. 9 is a flow diagram depicting stages in a method for employing a hand-held test meter according to various embodiments;

FIG. 10 is a block diagram of portions of a conventional hand-held test meter; and

FIG. 11 is a graph showing another simulated example of phase adjustment while sampling a current signal according to various exemplary embodiments.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. In addition, the term “in,” as used throughout this description does not necessarily require that one component or structure be completely contained within another, unless otherwise indicated.

Throughout the course of discussion, the symbols “i” or “I” each refer to electric current. Also throughout the course of discussion, ranges or intervals are denoted using square brackets for closed endpoints and parentheses for open endpoints, as is conventional in the mathematical art.

As used herein, the term “phase” refers to a time offset between two time-varying signals, expressed as the angle on a circle corresponding to the proportion between the time offset and the period of one of the signals. Phase can be measured in radians (0-2π rad) or degrees)(0-360° interchangeably. For example, if signal B is offset in time by 50% of the period of signal A, the phase of B with respect to A is π rad or 180°. Phases given herein without a unit (rad or °) are in radians.

Throughout this disclosure, mathematical computations taking phases as inputs are performed modulo 2π rad (360°) unless otherwise specified. For example, 180°+270°≡90° (mod 360°) since 180°+270°>360° and 180°+270°−360°=90°. Likewise, 180°−270°≡−90° (mod 360°). As a result, a phase can change from 300° to 60° either by adding 120° (300°+120°≡60° (mod 360°)), or by subtracting 240° (300°−240°=60°≡60° (mod 360°)).

As used herein, the term “crossing,” in the context of mathematical functions and curves, refers to a region of the function or curve in which the value of function or curve changes from above a selected value to below the selected value, or vice versa. For phases, crossings are determined modulo 2π. For example, when phase changes from 300° to 60°, the phase passes through a zero (0°) crossing if incremented by 120°, but not if decremented by 240°. Specifically, a “crossing” for purposes of this discussion is a change in phase of less than 180°, e.g., between successive measurements, as discussed below.

Various analytes can be detected by driving a time-varying voltage signal through the sample and measuring the phase difference between a resulting current signal and the time-varying voltage signal. The phase can be measured, for example, using a quadrature demodulator or lock-in amplifier.

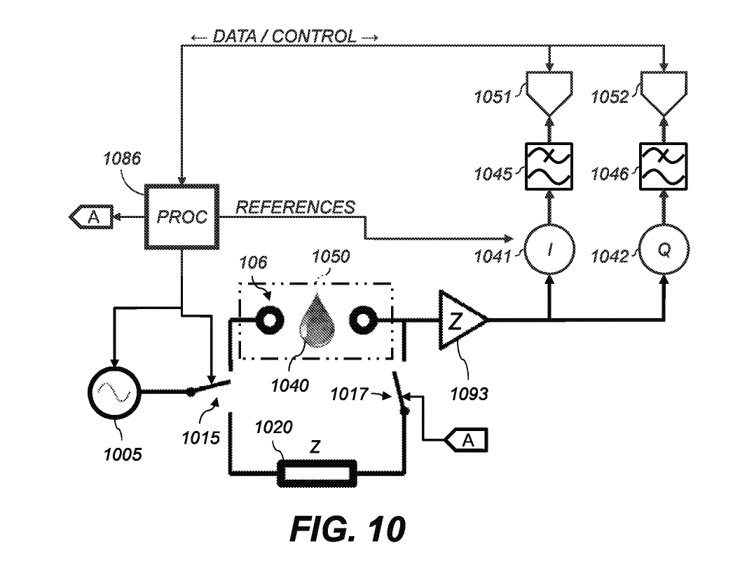

For purposes of providing sufficient background, reference is first made to FIG. 10, which depicts a block diagram of various portions of a conventional hand-held test meter used for measuring phase differences of current signals. More specifically, a processor 1086 causes a voltage supply 1005 to apply a sinusoidal voltage signal across a fluid sample 1040 (represented graphically as a droplet) applied to an analytical test strip 1050 (shown in phantom). The processor 1086 controls switches 1015, 1017 to pass the voltage signal through the sample 1040 or through a dummy load 1020. A transimpedance amplifier 1093 converts the resulting current through the fluid sample 1040 into a voltage signal.

That voltage signal is provided to quadrature demodulators 1041, 1042, which respectively provide in-phase and quadrature components of the measured signal. Each of the quadrature demodulators 1041, 1042 can include, e.g., a lock-in amplifier, or a multiplier for multiplying the input by a reference signal (“REFERENCES” from the processor 1086). The reference signal for the quadrature demodulator 1041 (“I” or in-phase) can be in phase with the voltage signal from the voltage supply 1005, and the reference signal for the quadrature demodulator 1042 (“Q” or quadrature) can be 90° out of phase with the voltage signal from the voltage supply 1005. The respective outputs of the quadrature demodulators 1041, 1042 are provided to respective low-pass filters 1045, 1046. The low-pass filters 1045, 1046 can, e.g., filter noise and unintentionally-introduced harmonics out of their inputs and provide respective DC voltages to respective analog-to-digital converters (ADCs) 1051, 1052. The ADCs 1051, 1052 provide digitized data of the in-phase and quadrature components, i.e., the outputs of the low-pass filters 1045, 1046, respectively, to the processor 1086. The processor 1086 uses the digitized values of the in-phase and quadrature components to determine the phase difference of the current signal with respect to the voltage signal.

It can be useful to measure or determine phase difference using fewer components than such conventional systems. Additionally, some quadrature demodulators require sinusoidal inputs. Accordingly, the voltage supply 1005 in such systems is required to include circuits for producing a sinusoid. Any deviations from a pure sinusoid can reduce the effectiveness of the measurements, so complex, expensive circuits for producing very pure sinusoidal signals (e.g., brick-wall low-pass filters) can be required. Other prior sampling techniques involve taking measurements at a high rate to capture the details of a waveform, but such techniques can have high power consumption and can reduce battery life of portable or hand-held test meters.

In general, portable test meters, such as hand-held test meters, for use with an analytical test strip in the determination of an analyte (such as glucose) in a bodily fluid sample (i.e., a whole blood sample) according to embodiments herein include a strip port connector, a signal-measurement circuit, and a processor.

The strip port connector is configured to receive an analytical test strip. The signal-measurement circuit is configured to apply a voltage signal across a sample applied to the analytical test strip, e.g., via the strip port connector, and detect a resulting current signal. The signal-measurement circuit provides data corresponding to the resulting current signal. The voltage signal is a periodic at an excitation frequency and the data are provided at a digitizing frequency and a selected phase with respect to the voltage signal. The term “periodic,” as used herein, refers to signals that substantially repeat over a selected time interval. “Periodic” does not imply that the signals are infinite in time extent. The selected phase is a phase of the time a measurement is taken with respect to the voltage signal. The selected phase is not related to the phase difference between the current signal and the voltage signal. For example, a given sequence of selected phases can be used to measure current signals having any phase difference from the voltage signal.

The processor is configured to repeatedly record one or more value(s) of the data and to alter the selected phase, so that a respective plurality of values is recorded at each of a plurality of phases. The processor determines a phase difference of the resulting current signal with respect to the voltage signal using the recorded values at the various selected phases.

Hand-held test meters according to embodiments of the present invention are beneficial in that they provide analyte determination via phase-difference information (such as hematocrit determination) in whole blood samples using simpler hardware configurations. Various embodiments can measure the hematocrit of the whole blood sample and then employ the measured hematocrit during analyte determination, e.g., glucose determination.

A problem solved by various embodiments is to accurately measure the phase difference of the fluid sample without using a lock-in amplifier or pure-sinusoid-producing circuit. This can be done using multiple measurements in a waveform according to various aspects. Using techniques described herein can beneficially reduce the physical size, weight, and cost of the electronics package in the test meter, as compared to some prior test meters that use phase-detection circuitry before an analog-to-digital converter (ADC). Omitting these electronics can also reduce power consumption of the electronics, thereby increasing battery life. The herein described invention is not limited to solving these problems.

The concepts discussed herein can easily be incorporated by one of sufficient skill into a hand-held test meter. One example of a test meter that can be suitably configured is the commercially available OneTouch® Ultra® 2 glucose meter from LifeScan Inc. (Milpitas, Calif.). Additional examples of hand-held test meters that can also be modified are found in U.S. Patent Application Publications Nos. 2007/0084734 (published on Apr. 19, 2007) and 2007/0087397 (published on Apr. 19, 2007) and in International Publication Number WO2010/049669 (published on May 6, 2010), each of which is hereby incorporated herein in full by reference.

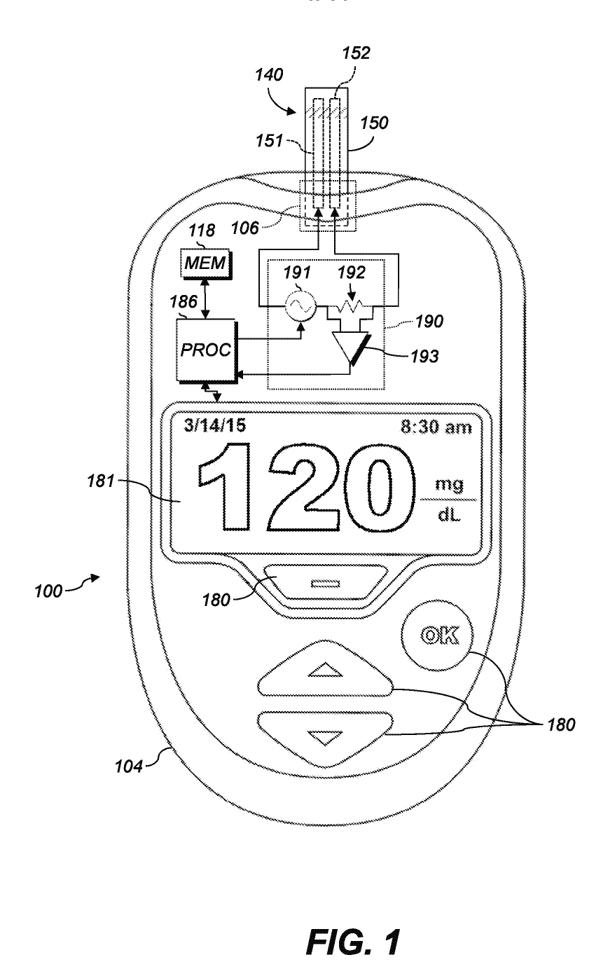

FIG. 1 is a simplified depiction of a hand-held test meter 100 and related components according to an exemplary embodiment. The hand-held test meter 100 includes a housing 104 and a strip port connector (also referred to throughout this description as an “SPC”) 106 that is configured to receive the analytical test strip 150, the latter being inserted into a port of the housing 104. The SPC 106 can include spring contacts arranged so that the analytical test strip 150 can be slid into the SPC 106 to electrically connect electrodes 151, 152 with a signal-measurement circuit 190. Alternatively, the SPC 106 can include pogo pins, solder bumps, pin or other receptacles, jacks, or other devices for selectively and removably making electrical connections. Specifically, in various embodiments, the strip port connector 106 is configured to operatively interface with a first electrode 151 and a second electrode 152 of the received analytical test strip 150, the first and second electrodes 151, 152 being disposed at least partly in a sample cell 140, such as an electrochemical cell.

The hand-held test meter 100 retains the signal-measurement circuit 190 and a processor 186. In an exemplary embodiment, the processor 186 causes application of an AC waveform across the sample cell 140 via the electrodes 151, 152, and causes concurrent measurement of a current through the electrodes 151, 152, e.g., using a current detector in the signal-measurement circuit 190.

In the example shown, the signal-measurement circuit 190 includes an AC voltage source 191 controlled by the processor 186. The AC voltage source 191 is connected to the first electrode 151. A current detector in the signal-measurement circuit 190 includes a resistor 192 in series between the electrode 152 and the AC voltage source 191. The voltage across the resistor 192 is directly proportional to the current through the AC voltage source 191 and the electrodes 151, 152. An amplifier 193 amplifies the voltage across the resistor 192 to provide a voltage signal to the processor 186 that is representative of current through the electrodes 151, 152. Other embodiments of current detectors, e.g., transimpedance amplifiers or Hall-effect current sensors, can be used instead of or in addition to the resistor 192 and the amplifier 193. Note that the term “amplifier” does not require that the amplifier 193 have >0 dB gain. The amplifier 193 can pass, attenuate, or boost signals.

Still referring to FIG. 1, the hand-held test meter 100 can also include a user interface including, e.g., a display 181 and one or more user interface buttons 180, for example, disposed on a facing surface of the housing 104. The display 181 can be, for example, a liquid crystal display or a bi-stable display configured to show a screen image. The exemplary screen image shown in FIG. 1 provides indications of glucose concentration (“120”) and of date and time (“Mar. 14 2015 8:30 am”), as well as a units indication (“mg/dL”). The display 181 can also present error messages or instructions to a user, for example, such as instructions for properly conducting a test (analyte determination).

The hand-held test meter 100 can also include other electronic components (not shown) for applying test voltages or other electrical signals to the analytical test strip 150, and for measuring an electrochemical response (e.g., plurality of test current values) and determining an analyte based on the electrochemical response. For purposes of clarity, the figures do not depict all such electronic circuitry. In an example, the processor 186 and the signal-measurement circuit 190 are configured to detect the presence of the fluid sample in the sample cell 140 of a received analytical test strip 150 and initiate a test sequence or assay based upon the detection of a fluid sample.

For the purposes described herein, the processor 186 can include any suitable microcontroller or micro-processor known to those of skill in the art. One exemplary microcontroller is an MSP430-series microcontroller that is commercially available from Texas Instruments, Dallas, Tex. USA. The processor 186 can include, e.g., a field-programmable gate array (FPGA) such as an ALTERA CYCLONE FPGA, a digital signal processor (DSP) such as a Texas Instruments TMS320C6747 DSP, or another suitable processing device adapted to carry out various algorithm(s) as described herein. The processor 186 can include signal-generation and signal-measurement functions, e.g., D/A converters, pulse-train generators, or A/D converters.

A memory block 118 of the hand-held test meter 100 includes one or more storage device(s), e.g., a code memory (such as random-access memory, RAM, or Flash memory) for storing, e.g., program firmware or software; a data memory (e.g., RAM or fast cache); or a disk (such as a hard drive). Computer program instructions to carry out suitable algorithm(s) can be stored in one of those device(s); this is referred to herein as “storing an algorithm.” The memory block 118 can also or alternatively be incorporated in the processor 186. A Flash or other nonvolatile memory in the memory block 118 can also contain, e.g., graphics to be displayed on the display 181, text messages to be displayed to a user, calibration data, user settings, or algorithm parameters.

The processor 186 can use information stored in the memory block 118 in determining an analyte, e.g., in determining a blood glucose concentration, based on the electrochemical response of analytical test strip. For example, the memory block 118 can store correction tables to adjust the determination of the analyte based on determined characteristic(s) of the analytical test strip 150.

Throughout this description, some embodiments are described in terms that would ordinarily be implemented as software programs. Those skilled in the art will readily recognize that the equivalent of such software can also be constructed in hardware (hard-wired or programmable), firmware, or micro-code. Given the systems and methods as described herein, software or firmware not specifically shown, suggested, or described herein that is useful for implementation of any embodiment is conventional and within the ordinary skill in such arts.

Once the analytical test strip 150 is interfaced with the hand-held test meter 100, or prior thereto, a fluid sample (e.g., a whole blood sample or a control-solution sample) is introduced into the sample cell 140 of the analytical test strip 150. The analytical test strip 150 can include enzymatic reagents that selectively and quantitatively transform an analyte in the fluid sample into another predetermined chemical form. For example, the analytical test strip 150 can be an electrochemical-based analytical test strip configured for the determination of glucose in a whole blood sample. Such an analytical test strip 150 can include an enzymatic reagent with ferricyanide and glucose oxidase so that glucose can be physically transformed into an oxidized form.

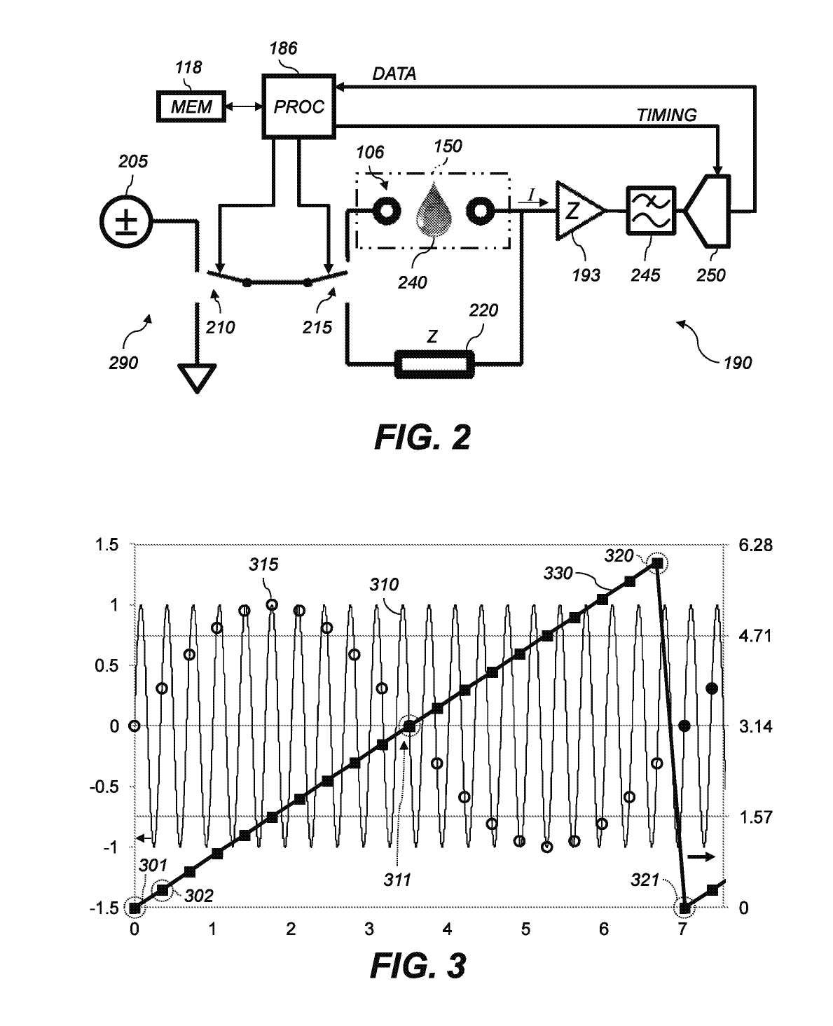

FIG. 2 is a simplified block diagram of a signal-measurement circuit 190 and related components in an exemplary hand-held test meter 100. The signal-measuring circuit 190 is configured to apply a voltage signal across a sample 240 (represented graphically as a droplet) applied to the analytical test strip 150. In various embodiments, the sample 240 is a fluid sample. In various of these embodiments, the sample 240 is a whole blood sample and the processor 186 is further configured to compute a level of hematocrit of the fluid sample based on the determined phase difference.

The voltage signal is a periodic at an excitation frequency. In various embodiments, the processor 186 is configured to provide a square wave as the voltage signal to the signal-measurement circuit 190. In this example, the processor 186 provides the square wave by controlling a switch 210 to repeatedly switch between ground and a voltage from a voltage source 205. This repeated switching provides the square wave, e.g., with a peak-to-peak voltage of 380 mV and a frequency of 75 kHz. Other waveforms and frequencies can also be used, and other types of pulse or waveform generators can be alternatively used in place of the switch 210.

The signal-measuring circuit 190 is also configured to detect a resulting current signal. In the example shown, the processor 186 controls a switch 215 (e.g., a TEXAS INSTRUMENTS TS5A3157) to selectively direct the voltage signal (e.g., the square wave) through either a calibration load 220 or the strip port connector 106 (represented graphically as two circles, standing for the contacts to the two electrodes). In various aspects, the calibration load 220 is designed so that its parasitic capacitance and susceptibility to aerial radio-frequency (RF) signals do not substantially affect the currents received, measured, or transduced by the amplifier 193. In various aspects, the switch 215 is designed so that its leakage when open does not substantially affect the currents received, measured, or transduced by the amplifier 193.

If the strip port connector 106 is selected, the voltage signal passes through the inserted analytical test strip 150 (shown in phantom) to the amplifier 193 (or other current-to-voltage converter). If the calibration load 220 is selected, the voltage signal passes through the calibration load 220, e.g., a precision resistor, to the amplifier 193, as discussed in more detail below. In various aspects, the amplifier 193 is a transimpedance amplifier configured to provide a detection-voltage signal corresponding to the resulting current signal. According to the exemplary embodiment, the amplifier 193 has a gain of 110000 V/A (Ω), although this value can be suitably varied. The amplifier 193 can also shift the DC level of the signal so that it is within the range of an analog-to-digital converter (ADC) 250.

The signal-measuring circuit 190 is further configured to provide data corresponding to the resulting current signal. In this exemplary embodiment, the ADC 250 samples the detection-voltage signal from the amplifier 193 and provides digital data (via the “DATA” signal path) to the processor 186. The processor 186 controls the ADC 250 (via the “TIMING” signal path) so that the ADC 250 provides data at a digitizing frequency and a selected phase with respect to the voltage signal. In various aspects, the ADC 250 provides the data of the detection-voltage signal to the processor 186. In various aspects, the processor 186 uses a timer to provide a trigger pulse when the timer elapses. The timer pulse triggers the ADC 250 to read data, so the ADC 250 acquires one sample per period of that timer. In various of these aspects, the processor 186 further uses the timer to provide a control signal, e.g., a square wave, to the switch 210 to provide the voltage signal, as discussed above.

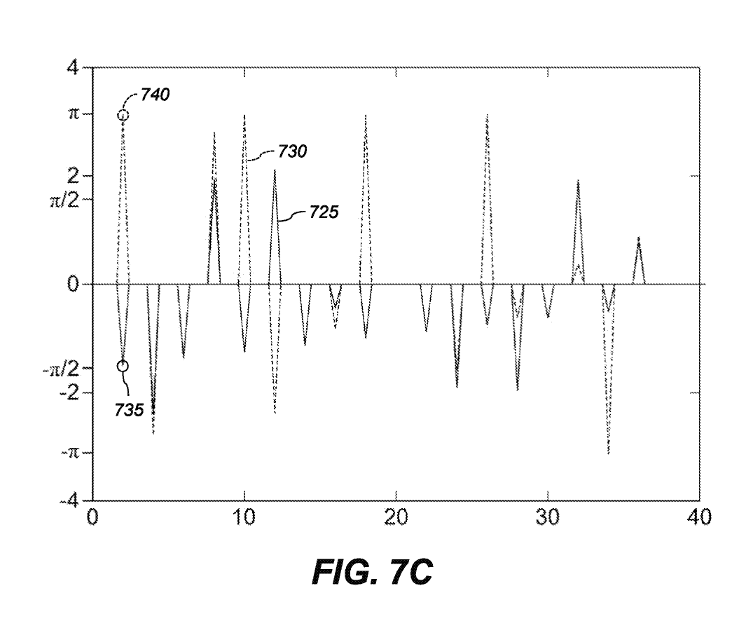

In various examples, a low-pass filter 245 is placed at the output of the amplifier 193, or is included in the amplifier 193. The low-pass filter 245 can be DC- or AC-coupled to the output of the amplifier 193. The low-pass filter 245 can also be a low-pass filter operable in the current domain and be placed at the input of the amplifier 193. Examples of the use of the low-pass filter 245 are discussed below with reference to FIGS. 7A-70.

In other examples, the output of the amplifier 193 is fed directly to an ADC 250 with substantially no intervening components. In these examples, passive components for performing impedance matching, balancing, or AC coupling can be used.

The transimpedance amplifier or other amplifier 193 can be connected to the ADC 250 through one or more non-switching component(s). Examples of non-switching components can include resistors, capacitors, inductors, and printed-circuit board traces or other fixed conductors. Exemplary switching components (not non-switching components) can include transistors, switches, and relays. In various aspects, connecting the amplifier 193 to the ADC 250 through non-switching components as described herein rather than through a lock-in amplifier or quadrature demodulator provides reduced parts count and thus reduced power consumption and increased battery life of the hand-held test meter 100.

In various embodiments, the processor 186 is configured to determine the phase difference using the voltage signal of a first frequency and a second voltage signal of a second frequency. The voltage signals can be provided successively or simultaneously. In an example, the analytical test strip 150 is an electrochemical-based analytical test strip configured for the determination of glucose in a whole blood sample. The fluid sample 240 is thus a whole blood sample. The first frequency can be in the range of about 10 kHz to about 25 kHz and the second frequency can be in the range of about 75 kHz to about 500 kHz. This permits, e.g., determining hematocrit of the whole blood cell using a frequency at which the response of the whole blood sample is affected by hematocrit. Likewise, glucose of the whole blood sample can be determined with a different frequency at which the response is affected by the presence of glucose.

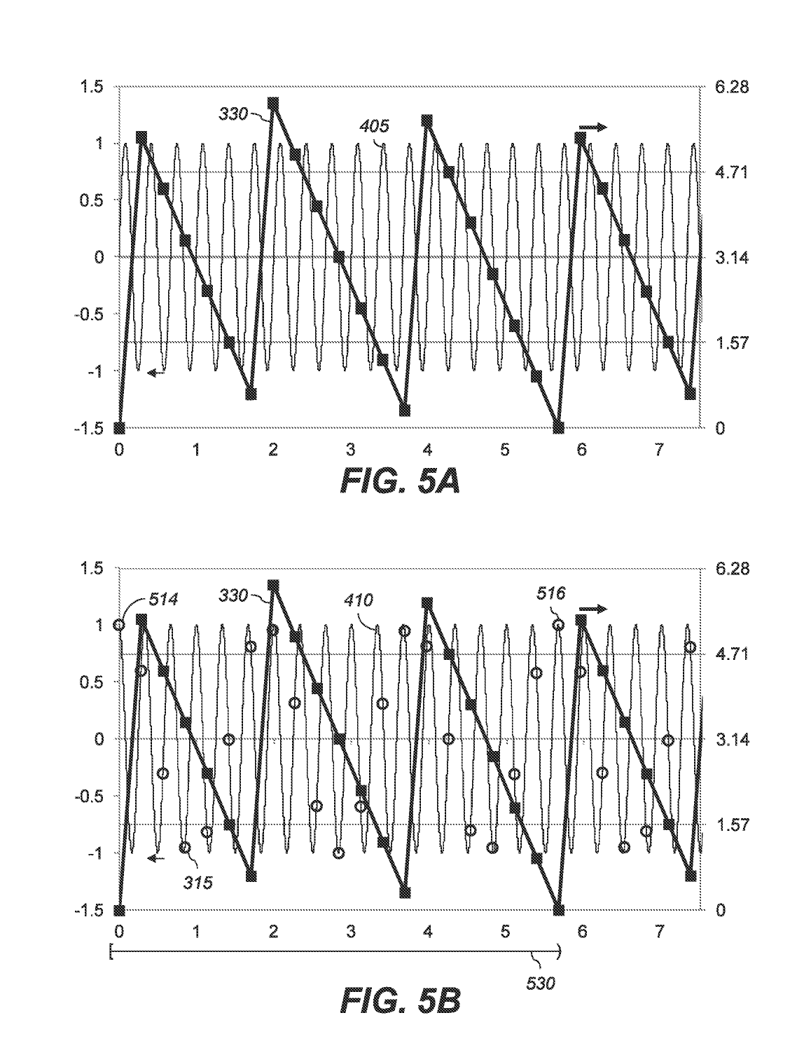

In various aspects, the hand-held test meter 100 includes the housing 104, FIG. 1. A square-wave generator 290 is disposed in the housing 104. In this example, the square-wave generator 290 includes the voltage source 205 and the switch 210, under control of the processor 186. In this paragraph, reference is made for exemplary purposes only to a voltage signal 405, FIG. 4A, and a resulting current signal 410, FIG. 4B. The square-wave generator 290 is configured to generate the square-wave voltage signal 405 and to supply the generated square-wave voltage signal 405 to an electrode 151, FIG. 1, of the analytical test strip 150 inserted into the hand-held test meter 100.

The amplifier 193 is a two-stage transimpedance amplifier disposed in the housing 104. The two-stage transimpedance amplifier 193 is configured to receive from the analytical test strip 150 the resulting current signal 410 (marked I in FIG. 2). The resulting current signal 410 originated from the square-wave voltage signal 405. That is, the current signal 410 arises when the voltage applied to the analytical test strip 150 by the square-wave generator 290 causes electric charge to flow through the sample cell 140, FIG. 1, or the fluid sample 240. This flowing electric charge is the resulting current signal 410. The magnitude of the resulting current signal 410, or the phase difference between the resulting current signal 410 and the square-wave voltage signal 405, can be correlated with properties of the analytical test strip 150 or of the fluid sample 240 in the sample cell 140.

The two-stage transimpedance amplifier 193 can include, e.g., two current-to-voltage stages in parallel, or two current-feedback op amps in series, or other combinations of gain stages. The two-stage transimpedance amplifier 193 can convert current to voltage in the first stage and amplify voltage in the second stage, or perform other combinations of current-to-voltage conversion, current amplification, or voltage amplification.

The memory block 118 is connected to the processor 186 and stores a digital filtering algorithm (i.e., stores computer program instructions to be carried out by processor 186 to perform the steps of a digital filtering algorithm). The memory block 118 and the processor 186 are both disposed in the housing 104. The processor 186 is configured to automatically execute the digital filtering algorithm (i.e., to automatically retrieve the stored computer program instructions from the memory 118 and execute them). The processor 186 executes the algorithm to recover a fundamental phase and magnitude from the resulting current signal. The algorithm can include performing a Fourier transform of the resulting current signal 410 and extracting the phase or magnitude corresponding to the lowest-frequency sinusoidal component above DC (i.e., >0 Hz). An example of Fourier transforms is discussed below with reference to FIGS. 7A-7C.

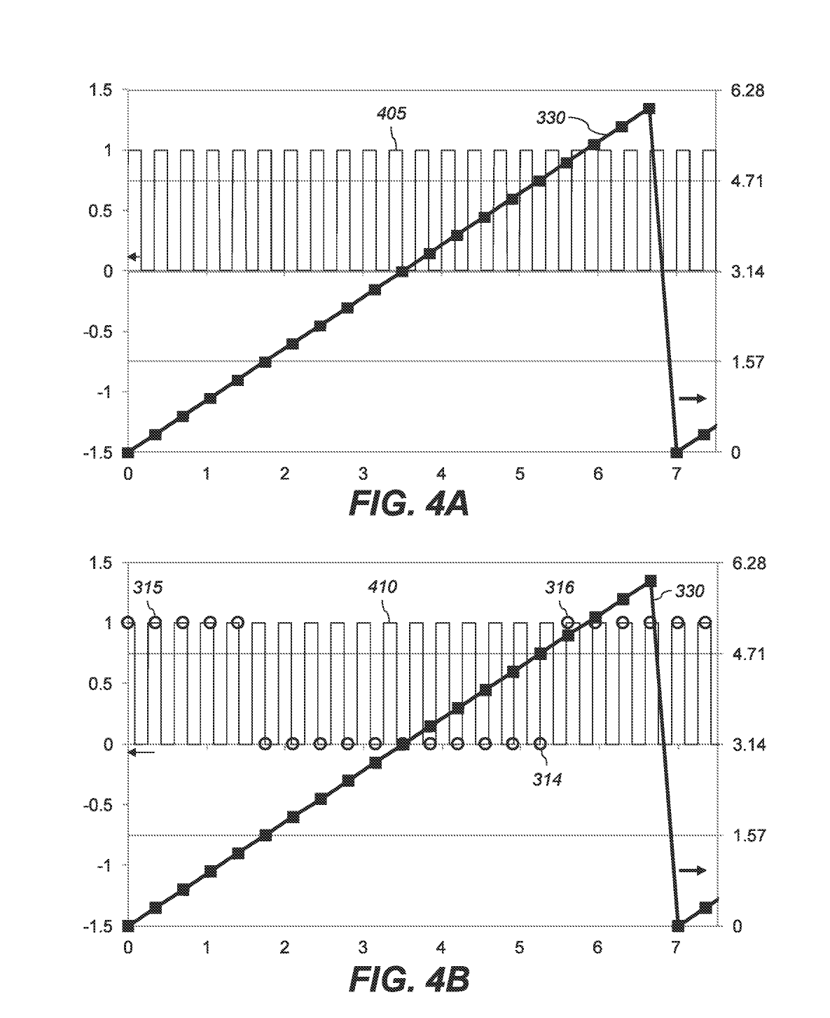

FIG. 3 is a graph showing a simulated example of phase adjustment while sampling a current signal 310. The abscissa shows time in arbitrary units (a.u.). In an example, the current signal 310 is a 3 Hz signal and the abscissa shows time in seconds. The left-hand ordinate shows value of the current signal 310 (a.u., e.g., V or percent amplitude), and the right-hand ordinate shows phase in rad, as is discussed below with reference to a curve 330. In this example, the current signal 310 is sinusoidal for clarity of exposition. Circles indicate points 315 (for clarity, only one of the circles is labeled) on the current signal 310 at which measurements are taken by the ADC 250 under control of the processor 186. In this example, the current signal 310 is measured at time intervals equal to 105% (1.05×) the period of the current signal 310. As a result, each successive measurement is at a different selected phase with respect to the voltage signal. In this example, the current signal 310 is assumed to be in phase with the voltage signal, i.e., to have a phase difference of zero. However, this is not required, as is discussed below.

The selected phase at each measurement is shown by the curve 330, which has values in radians indicated on the right-hand ordinate. As shown, a first-measurement phase 301 is 0 rad with respect to the current signal 310, and thus with respect to the voltage signal of which the current signal is a result (in this example using a phase difference of 0 rad). A second-measurement phase 302 is π/10 rad, a third-measurement phase π/5 rad, and so forth. Each successive measurement is π/10 farther in phase than the measurement before it. Therefore, an eleventh-measurement phase 311 is π rad, and a twentieth-measurement phase 320 is 19π/10. The curve 330 thus has a π-crossing substantially at the measurement corresponding to the phase 311. The twenty-first-measurement phase 321 is 20π/10=2π=0 rad. The curve 330 has a zero crossing substantially at phase 321, since selected phase is incremented modulo 2π to pass from the measurement phase 320 to the measurement phase 321.

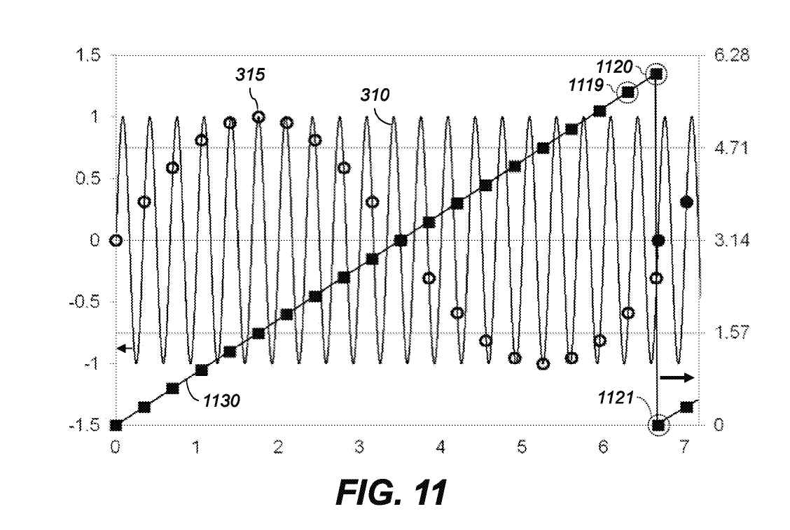

The 20 measurements from the measurement 301 to the measurement 320 are taken over the course of 21 cycles of the current signal 310. When the current signal 310 is substantially stable over those 21 cycles, information about a full cycle is obtained. To measure that information in a single cycle would have required sampling approximately 19 times faster, increasing power consumption and possibly reducing measurement accuracy (due to switching transients being closer in time to measurements). In this technique, the constant phase increment can be applied a plurality of times to take successive 20-measurement sets over successive groups of 21 cycles of the current signal 310. In this example, a first group of 20 samples is shown using hollow circles for the measurement points 315. The first two samples of a second group of 20 samples are shown using solid circles for the measurement points 315. Spacing between groups of samples is discussed below with reference to FIG. 11.

The processor 186 is configured to repeatedly record one or more value(s) of the data and to alter the selected phase, so that a respective set of one or more value(s) is recorded at each of a plurality of selected phases. In the example shown in FIG. 3, the processor 186 records a single value at each selected phase (e.g., 0 rad at the measurement 301) and then alters the selected phase (e.g., to π/10 rad at the measurement 302). The processor 186 is further configured to determine a phase difference of the resulting current signal 310 using the respective sets of value(s) at the selected phases.

The processor 186 can be further configured to provide a representative value at each of the plurality of selected phases (phases of measurements that are taken) using the respective set of value(s). Each representative value can be equal to the single measured value. Alternatively, the processor 186 can record respective values over two full cycles of the selected phases (here, 42 periods of the current signal 310) and provide the average of the two values at each selected phase as the representative value for that selected phase. The processor 186, in this example, is configured to determine a phase difference of the resulting current signal with respect to the voltage signal using the representative values.

FIG. 11 is a graph showing another simulated example of phase adjustment while sampling a current signal 310. The ordinates are as in FIG. 3. A group of twenty measurement points 315 are shown as open circles. Measurement points in a following group of twenty measurement points are shown as solid circles.

A curve 1130 shows the selected phase (squares) of each successive measurement point 315 (circles). This example shows in-sequence undersampling at 105% of a 3 Hz signal, with axes in seconds. The nineteenth measurement has a selected phase 1119 of 18π/10, the twentieth measurement has a selected phase 1120 of 19π/10, and the twenty-first measurement has a selected phase 1121 of 20π/10=0 (mod 2π), as in FIG. 3. Like FIG. 3, the time between the measurement at the phase 1119 and the measurement at the phase 1120 is 0.35 s (105% of a 3 Hz period). However, unlike FIG. 3, the time between the measurement at the selected phase 1120 (rightmost hollow circle) and the measurement at the selected phase 1121 (leftmost solid circle) is not 0.35 s. Instead, it is 16% ms, 5% of a 3 Hz period. 20 measurements define nineteen 105% intervals between measurements. The measurement phase thus increments to a total of 19*105%=1995% over those 20 measurements. The remaining 5% of the period to bring the phase to an even multiple of 100% (i.e., in phase with the current signal 310) is the 5%, or 16⅔ ms.

In this example, 20 measurements are taken over the course of 20 cycles of the current signal 310. When the current signal 310 is substantially stable over those 20 cycles, information about a full cycle is obtained. To measure that information in a single cycle would have required sampling approximately 20 times faster, increasing power consumption and possibly reducing measurement accuracy. Constant phase increment within sets and a smaller phase increment between sets can be applied a plurality of times to take successive 20-measurement sets over successive groups of 20 cycles of the current signal 310. Similarly, the processor 186 can record respective values over two full cycles of the selected phases (e.g., 41 periods of the current signal 310) and provide the average of the two values at each selected phase as the representative value for that selected phase.

Throughout this disclosure, the separation between the end of one group of measurements and the beginning of the next group of measurements can include whatever time is required to bring the measurements back into phase with the signal being measured, e.g., the current signal 310. This separation can also the time required for one or more complete cycles of the current signal 310 or other signal being measured. The separation can be the same between groups of measurements as within a group of measurements, or different.

FIGS. 4A and 4B show a simulated example of measuring data used for determining phase differences. The respective axes are as in FIG. 3. In this example, a voltage signal 405 and a current signal 410 are square waves from 0 to +1 (a.u.). Other amplitudes, offsets, waveforms, or frequencies of the voltage signal 405 (and thus of the current signal 410) can also be used.

FIG. 4A shows the voltage signal 405 according to this example. The curve 330 shows the selected phases at a plurality of measurement points with respect to the voltage signal 405, as discussed above with reference to FIG. 3. In this example, constant selected-phase separation is used, as discussed above. FIG. 4B shows the exemplary current signal 410. In this example, the current signal 410 is 90° (π/2 rad) out of phase with the voltage signal 405. Specifically, a cycle of the current signal 410 begins 90° before (≡270° after) a cycle of the voltage signal 405 (e.g., a phase difference of 90°). Points 314, 315, 316 (for clarity, not all measurement points are labeled) show that 20 measurements are taken over 21 cycles of the voltage signal 405. The curve 330 shows that each measurement corresponds to a different selected phase with respect to the voltage signal 405.

In this example, an approximate phase difference can be determined by inspection of transitions. The processor 186 can determine between which points the measured current signal 410 transitions from 0 to 1. In this example, that transition is between points 314 and 316. The point 314 is at a selected phase on the curve 330 of 3π/2 rad, and the point 316 is at a selected phase on the curve 330 of 8π/5 rad. A 0-to-1 transition of the voltage signal occurs at a phase of 0 rad. Accordingly, the phase difference of the current signal 410 with respect to the voltage signal 405 is on the range [3π/2,8π/5]=[270°,288° ] (modulo 2π). This corresponds to the simulated phase difference of −90°≡270° (mod 2π). More accurate measurements of phase can be determined using Fourier transforms, as described below.

The technique shown in FIG. 4B is referred to herein as “in-sequence undersampling.” In this technique, the processor 186 is configured to record a selected value (e.g., the measurement 301, FIG. 3) at a selected first phase (e.g., 0 rad). The processor 186 then alters the selected first phase by a selected phase offset (e.g., π/10 rad) to provide a second phase different from the first phase (e.g., π/10 rad). The processor 186 subsequently records a value (e.g., measurement 302, FIG. 3) at the provided second phase.

In various embodiments, such as those illustrated in FIGS. 3-4B, the selected phase offset is greater than zero. The second phase is thus greater than the selected first phase (mod 2π). Note that providing the selected measurement phase 321 from the selected phase 320, both FIG. 3, is an adjustment by a phase offset greater than zero since the adjustment is performed modulo 2π: 19π/10+π/10=20π/10≡0 (mod 2π). In other embodiments, the selected phase offset is less than zero.

In at least one example, the processor selects the first and second (and subsequent) selected phases by multiplying the period of the voltage signal 405 by a fixed multiplier. FIGS. 3 and 4B show a multiplier of 105%=1.05. For example, for a 75 kHz voltage signal 405, measurements can be taken at a rate of [1.05/(75×10³)]⁻¹≈71.42857 kHz to perform 20 measurements in 21 cycles of the voltage signal 405 (21=1.05×20; the number of cycles of the voltage signal 405 times the multiplier can be an integer). In various examples using multipliers >100%, the selected phase offset is positive (e.g., +π/10 for a multiplier of 105%).

Measurements can also be taken more or less frequently, e.g., at multipliers of 102.5% (40 measurements over 41 cycles of the voltage signal 405) 205% (20 measurements over 41 cycles of the voltage signal 405) or 305% (20 measurements over 61 cycles of the voltage signal 405). In another example, 53 measurements can be taken in 52 cycles of the voltage signal 405 (multiplier≈101.9%). The measurement sequence can also decrease in selected phase rather than increasing. For example, a multiplier of 95% results in taking 20 measurements in 19 cycles of the voltage signal 405, with the selected phase of each successive measurement −π/10 (mod 2π) compared to the last, rather than +π/10 for a multiplier of 105%. In this and other examples using multipliers <100%, the selected phase offset is negative, i.e., less than zero.

In various aspects, the memory block 118, FIG. 1, is configured to store a sequence of a plurality of selected phases and to provide the sequence to the processor 186. The processor 186 is configured to alter the selected phase to successive values in the sequence. In this way, the processor 186 successively records a respective value at each of a plurality of selected phases in the phase sequence. In the example shown in FIGS. 4A and 4B, the phase sequence is [0, π/10, 2π/10, 3π/10, . . . , 19π/10], and can be repeated as many times as desired to capture measurements. In this example, the sequence includes exactly one π-crossing, as discussed above with reference to the measurement 311, FIG. 3. A multiplier of 95% also provides a sequence [0, −π/10, . . . , −19π/10]≡[0, 19π/10, 18π/10, . . . , π/10] (mod 2π). This sequence also includes exactly one π-crossing since, as defined above, the transition from 0 to 19π/10 is >180° and thus not a crossing.

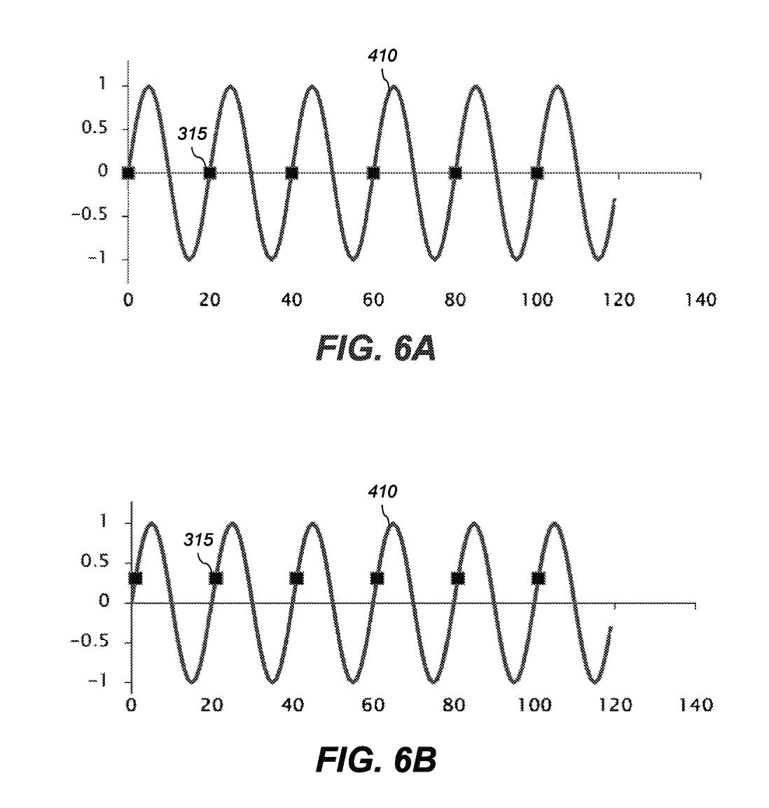

FIGS. 5A and 5B show a simulated example of a technique referred to herein as “out-of-sequence undersampling.” For clarity of exposition, these figures show a sinusoidal voltage signal 405 and a sinusoidal current signal 410 with measurements taken at points 315 (not all are labeled). In this example, constant selected-phase separation is used, as discussed above. As the curve 330 shows, the sequence includes more than one π-crossing. In this example, a multiplier of 85% is used. The resulting sequence is

[0,−3π/10,−6π/10, . . . ,−57π/10]

≡[0,17π/10,14π/10,11π/10,8π/10,5π/10,2π/10,

19π/10,16π/10,13π/10,π,7π/10, . . . , . . . ,3π/10] (mod 2π)

The sequence has 20 points, and those points are spread over 17 cycles of the voltage signal 405. The first π-crossing in the sequence is between 11π/10 mod 2π and 8π/10 mod 2π, the second is between 13π/10 mod 2π and 7π/10 mod 2π, and the third and last π-crossing in the sequence is between 12π/10≡−48π/10 and 9π/10≡−51π/10 as shown in the sequence excerpt above. A range marker 530 shows the extent of the sequence, which includes a measurement 514 at 0° selected phase and excludes a subsequent measurement 516 at 0° selected phase.

In various examples, when the sequence has a regular step between elements (e.g., −3π/10≡17π/10 (mod 2π) per sequence element in the example above), the sequence can be converted to specific sampling times by determining the sampling spacing. The sampling spacing in seconds is the product of the regular step in radians (or degrees) and the period in seconds of the voltage signal 405, divided by 2π (360°). This converts radians (degrees) into seconds. Measurements are taken at times corresponding to integer multiple(s) of the sampling spacing.

In other examples, e.g., when the sequence does not have a regular step between elements, the sequence can be converted to specific sampling times Tₙ. The values φₙ of the sequence can be indexed by integers n, 0≦n<d, for integer d; in the example above, d=20. Each value φₙ is wrapped to the range [0,2π], resulting in wrapped phases Ψₙ. Each Ψₙ is multiplied by the period t of the voltage signal 405 and divided by 2π to convert it to the corresponding time offset Sₙ. Let Δₖ=Sₖ−Sₖ₋₁ for 0<k<d. Then, the sampling time Tₙ, 0<n<d, is (selecting an initial value for T₀, e.g., 0):

$${T}_{n} = \{ \begin{matrix}{T}_{n - 1} + {Δ}_{n} , & {Δ}_{n} \geq 0 \\ {T}_{n - 1} + {Δ}_{n} + t , & {Δ}_{n} < 0\end{matrix} \qquad ( 3 )$$

An example is given in Table 1. This example uses a period t=2 s for the voltage signal 405 for ease of explanation. Only the first eight values of the sequence are shown, even though the sequence has 20 values as given in the example above.

TABLE 1

| | Sequence value | Wrapped phase | Time offset | | |
|---|---|---|---|---|---|
| n | Φₙ (rad) | Ψₙ (rad) | Sₙ (s) | Δₖ (s) | Tₙ (s) |
| 0 | 0 | 0 | 0 | N/A | 0 |
| 1 | −3π/10 | 17π/10 | 1.7 | 1.7 | 1.7 |
| 2 | −6π/10 | 14π/10 | 1.4 | −0.3 | 3.4 |
| 3 | −9π/10 | 11π/10 | 1.1 | −0.3 | 5.1 |
| 4 | −12π/10 | 8π/10 | 0.8 | −0.3 | 6.8 |
| 5 | −15π/10 | 5π/10 | 0.5 | −0.3 | 8.5 |
| 6 | −18π/10 | 2π/10 | 0.2 | −0.3 | 10.2 |
| 7 | −21π/10 | 19π/10 | 1.9 | 1.7 | 11.9 |

In various embodiments, the excitation frequency corresponds to an excitation period t and the sequence consists of values φₙ. Each value φₙ in the sequence is substantially equal to an aim value θₙ:

$${Θ}_{n} = [ ( n \cdot tp ) mod t ] \times \frac{2 π}{t} \qquad ( 2 )$$

where p is the multiplier, or in general a selected percentage p=^{k}/_{d}<100%, e.g., 85%. Positive integers k and d, k<d, define the percentage; e.g., k=17 and d=20 gives p=85%. Integers 0≦n<d or 0<n≦d index the elements of the sequence. In various aspects, such as 85%, k is an odd prime number (e.g., 17). As discussed above with reference to FIG. 11, the aim values can be adjusted for non-constant separation. For example, aim values can be decreased each successive set of measurements to take into account a 5% separation between sets instead of a 105% sequence between sets.

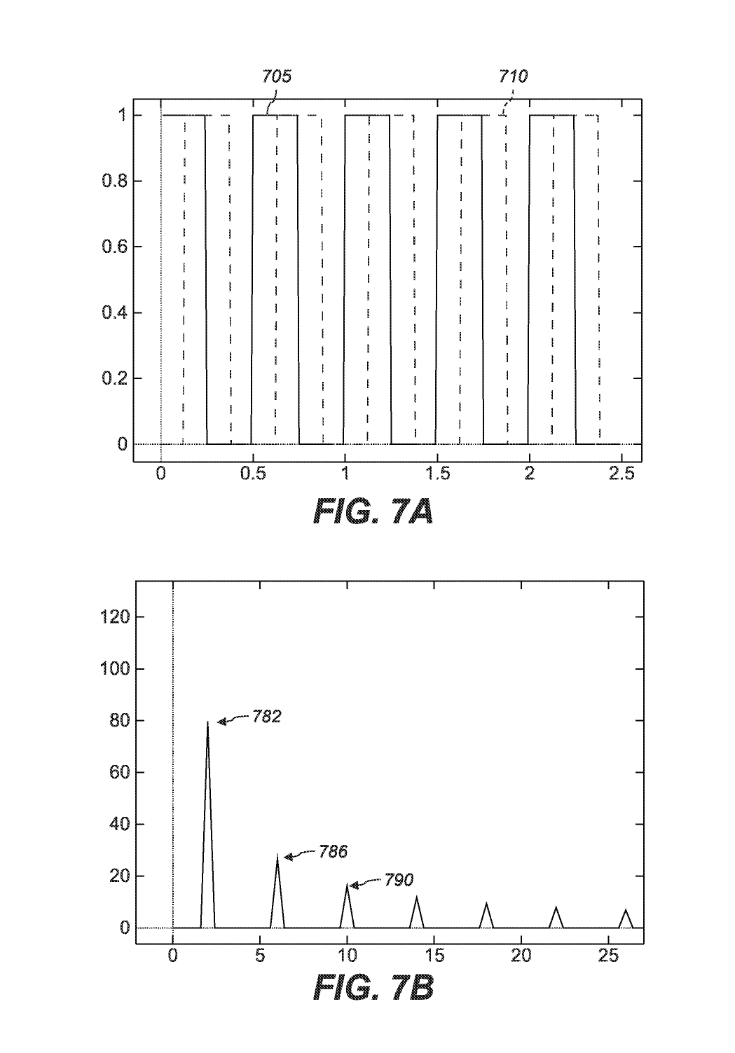

FIGS. 6A and 6B show a simulated example of a technique referred to herein as “in-sequence phase shifting.” In these figures, the abscissa is time (a.u.) and the ordinate is current-signal magnitude (a.u.). In this technique, the processor 186 is configured to first successively record a plurality of values in the respective set of value(s) for a selected first phase. This is shown in FIG. 6A, in which the selected first phase is 0°. Measurements are taken at points 315 indicated by squares. For clarity, not all the points 315 are labeled. The processor 186 then alters the selected first phase to a selected second phase, and successively records a plurality of values in the respective set of value(s) for the selected second phase. This is shown in FIG. 6B, in which the selected second phase is approximately 18°. Compared to FIG. 6A, the points 315 are later in time in FIG. 6B. Each point 315 in FIG. 6A represents a measurement taken substantially at the selected first phase, and each point 315 in FIG. 6B represents a measurement taken substantially at the second phase. As discussed above, the selected first and second phases can be used regardless of the phase difference of the current signal 410. In various examples, the first and second phases are the first two elements in a phase sequence that has exactly one π-crossing. “Out-of-sequence phase shifting” can also be performed as for in-sequence phase shifting, but with the sequence of phases having more than one π-crossing.

In various embodiments, the processor 186 is configured to determine a respective average of the recorded plurality of values in each of the respective set of value(s) for the respective one of the plurality of selected phases. For example, the processor 186 can average the values measured at the six points 315 in FIG. 6A to determine the average value at a selected phase of 0°, and can average the values measured at the six points 315 in FIG. 6B to determine the average at a selected phase of 18°. The average can be, e.g., an arithmetic, geometric, quadratic (RMS), or harmonic mean, a median, or a mode. The processor 186 can then determine the phase difference of the resulting current signal by applying a Fourier transform to the determined averages.

In various embodiments, the processor 186 is adapted to determine the phase difference of the current signal 410 with respect to the voltage signal 405 by applying a Fourier transform to the sets of recorded value(s) at the plurality of selected phases. The Fourier transform can be discrete (DFT or FFT) or continuous. Other transforms between time and frequency domains can also be used. The phase difference can be determined using only the fundamental frequency components of the voltage signal 405 and the current signal 410, or other components. Other ways of determining the phase difference of a signal or signal component with a particular frequency can be used, e.g., suitably-designed finite impulse response (FIR) or infinite impulse response (IIR) filters.

FIGS. 7A-7C show a simulated example of a Fourier transform. In FIG. 7A, the abscissa is time (sec.) and the ordinate is signal value (a.u.). FIG. 7A shows two exemplary square waves of 50% duty cycle and period 0.5, i.e., frequency 2 Hz. A reference waveform 705 has a rising edge (0→1 transition) at time 0. A response waveform 710 has a rising edge at time 0.125. The response waveform 710 therefore lags the reference waveform by 90° (0.125/0.5×360°).

FIG. 7B shows the amplitude portion of the Fourier-transform frequency spectrum of the reference waveform 705. The abscissa is frequency (Hz) and the ordinate is amplitude (a.u.). A peak 782, at 2 Hz, represents the fundamental frequency of the reference waveform 705. A square wave contains energy at all odd harmonics, i.e., at the fundamental frequency times 3, 5, 7, . . . . The harmonics decrease in amplitude as frequency increases. For example, a peak 786 represents the 6 Hz (2 Hz×3) term, and a peak 790 represents the 10 Hz (2 Hz×5) term. This plot can be produced by taking the discrete Fourier transform (e.g., fast Fourier transform or FFT) of the reference waveform 705 at a sampling interval of 0.01 s, which produces complex-valued results. The plot shown in FIG. 7B is the complex modulus (complex magnitude) of each point in the transform results.

In various aspects, the amplifier 193 can include or be connected in series with the low-pass filter 245, both FIG. 2, that attenuates high frequencies in the input. For example, a low-pass filter can reduce aliasing. It is well known in the signal processing art that, for a given sampling rate and signal frequency, certain harmonics of the signal being measured will appear as if they were other harmonics. An example of aliasing is a 75 kHz square wave measured at a 1.5 MHz sampling rate. The square wave has all odd harmonics, as illustrated in FIG. 7B. When sampling the nth harmonic discretely, image frequencies of

|n×75k−m×1.5M|

will appear in the measured signal, for integer n, m. In this example, for n=19, 21, 39, 41, . . . , and m=1, the image frequencies are 75 kHz. Consequently, starting at the 19th harmonic, frequency content from some higher harmonics will be measured as if it were the 75 kHz fundamental frequency.

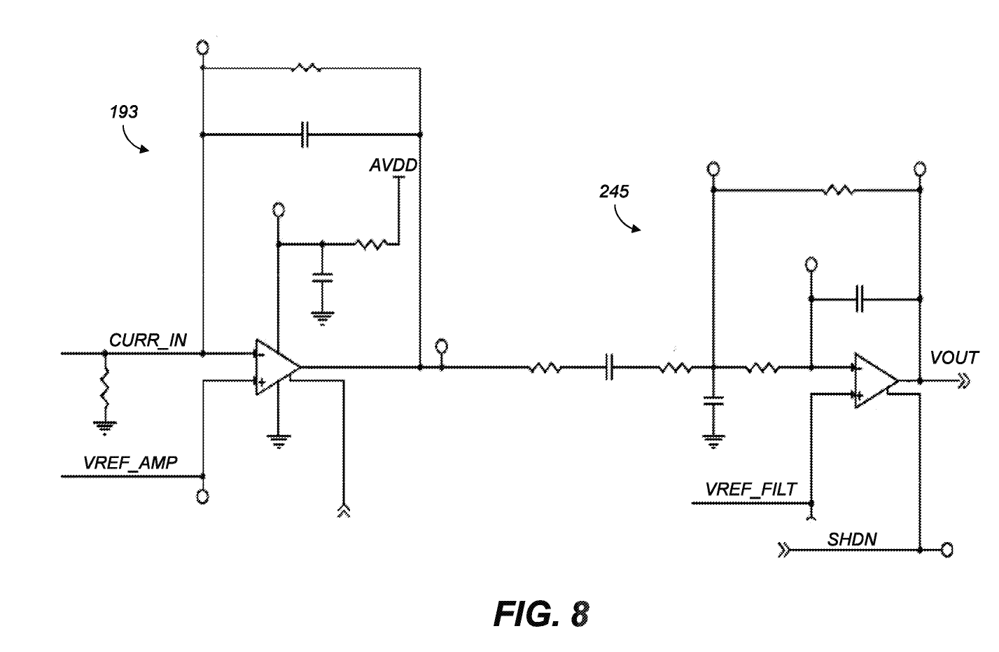

To mitigate error arising from this effect, the low-pass filter 245 can be designed to have a selected degree of attenuation at a desired frequency. For example, using the ADC 250, FIG. 2, the 19th harmonic and all higher harmonics can be attenuated below 1 LSB of the ADC 250. For a 12-bit linear ADC 250, the attenuation should be <1/(2¹²), i.e., <−72 dB. The low-pass filter 245 can be a 3^{rd}-order low-pass filter with a knee frequency less than about 90 kHz to provide this order of attenuation. The remaining harmonics in the signal can be discriminated using the Fourier transform, as discussed above. An example of the low-pass filter 245 is shown in FIG. 8, discussed below.

FIG. 7C shows the phase portion of the Fourier-transform frequency spectrum of the reference waveform 705. The Fourier transform can be performed as described above with reference to FIG. 7B. In FIG. 7C, the abscissa is frequency (Hz) and the ordinate is phase (rad). A phase 725 (solid trace) of the reference waveform 705 is shown on the same time scale as a phase 730 (dashed trace) of the response waveform 710. In this example, the abscissa extends from 0 Hz (DC) to 40 Hz, the 20th harmonic of the 2 Hz exemplary signals. The low-pass filter 245 can be used as discussed above to filter out harmonics at and above the 20th.

In this example, a peak 735 of the phase 725 and a peak 740 of the phase 730 correspond to the fundamental frequency of 2 Hz. The peak 735 indicates that the phase of the 2 Hz fundamental component of the reference waveform 705 (representative of, e.g., the voltage signal 405) is −π/2)(−90°. This is because a phase of 0° corresponds to a cosine, i.e., a waveform with a peak centered at time 0. The reference waveform 705 has its peak centered at 0.125 s, i.e., π/2 rad. Similarly, the peak 740 indicates that the phase of the 2 Hz fundamental component of the response waveform 710 (representative of, e.g., the current signal 410) is π(180°). The negative peak of the response waveform 710 is at time 0, so the fundamental is 180° out of phase with a cosine. Note that, for periodic signals, +180° and −180° are equivalent.

In this example, the processor 186 can compute the Fourier transform of the reference waveform 705 and the response waveform 710, which it can measure, e.g., as described above with reference to FIG. 2. The processor 186 determines the frequency of the reference waveform 705, so the processor 186 can retrieve the phases corresponding to that frequency from the Fourier transforms. The processor 186 can then determine the phase difference. In this example, the phase difference is π−−π/2=3π/2 rad. The processor 186 can then wrap this value to the range [−π,π] as is conventional in the signal processing art, yielding −π/2 as the phase difference between the reference waveform 705 and the response waveform 710.

Referring back to FIG. 4B, there is shown an example of in-sequence undersampling with a multiplier of 105%. The current signal 410 is 90° out of phase with the voltage signal 405. Assuming the abscissas of FIGS. 4A and 4B are time in seconds, the voltage signal 405 has an excitation frequency of 3 Hz, so the current signal 410 also has a frequency of 3 Hz in this example. The measurement frequency is ˜2.857 Hz. To determine the phase difference of the current signal 410, a Fourier transform can be performed of the measured values at the points 315. More than one set of 20 measurements can be taken. For example, 10 sets of 20 measurements (200 measurements) can be taken over 10 groups of 21 cycles of the signal (210 cycles). The 10 measurements for each selected phase can be averaged. Alternatively, the successive values at the 200 points 315 measured in this example can be provided to the Fourier transform process. This can provide information about higher frequencies since, in a conventional discrete Fourier transform process, the number of frequencies present in the frequency spectrum is proportional to the number of input points.

In an example, averaging is performed. That is, the points 315 corresponding to a given selected (measurement) phase are averaged. The result is, e.g., a set of 20 averaged points. That set of 20 averaged points is Fourier-transformed to determine the phase difference of the current signal 410.

In an example, a discrete Fourier transform is used. As is known in the signal-processing art, the discrete Fourier transform operates independently of sampling frequency. The sampling frequency is used to correctly assign the transformed coefficients to specific frequencies. In this example, the measurement frequency is ˜2.857 Hz, so the sampling period is 0.35 s. Accordingly, in this example, the set of 20 averaged points can be treated as points spaced by 0.35 s, as they were actually measured. Alternatively, the set of 20 averaged points can be treated as points spaced by 16⅔ms (=1/3 Hz/20), as if they had been measured sequentially at 20 equally-spaced intervals on a single cycle of the 3 Hz current signal 410. Appropriate calculations can be performed on the Fourier-transform results depending on which is chosen.

FIG. 8 shows exemplary circuits that can be used in various embodiments. FIG. 8 shows a transimpedance amplifier as the amplifier 193. The amplifier 193 includes some filtering, e.g., due to the capacitor in the op-amp feedback path. The amplifier 193 is AC-coupled to the low-pass filter 245, e.g., a 2^{nd}-order Bessel multifeedback filter. Other filters can be used instead of or in addition to the low-pass filter 245.

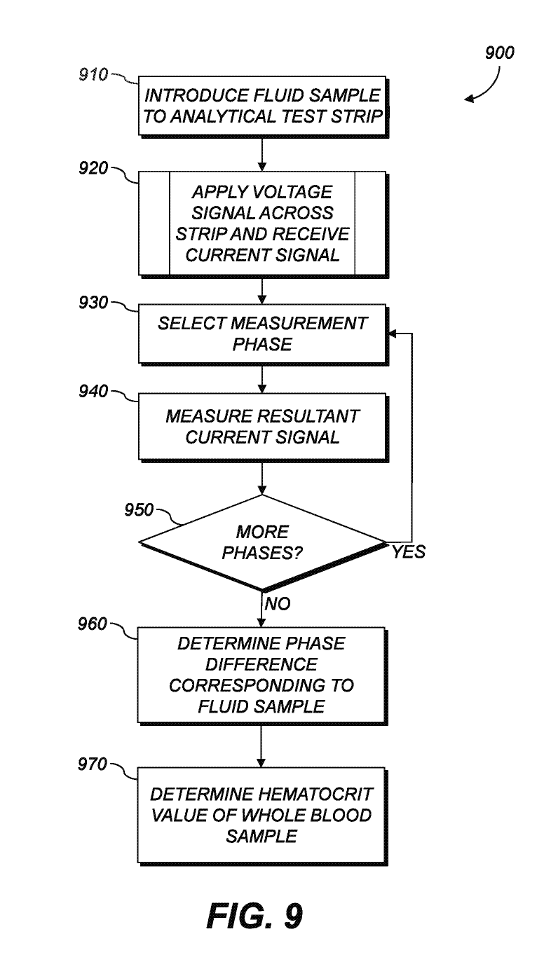

FIG. 9 is a flow diagram depicting stages in a method 900 for employing a hand-held test meter and analytical test strip (e.g., an electrochemical-based analytical test strip). Reference is made to various components described above for exemplary purposes. Methods described herein are not limited to being performed only by the identified components.

The method 900, at step 910, includes introducing a fluid sample, e.g., a whole blood sample to the analytical test strip (e.g., into a sample cell of the analytical test strip).

At step 920, a periodic voltage signal is applied across the test strip and a resultant current signal is received. The current signal can be received, e.g., by the amplifier 193. The amplifier 193 can provide the voltage signal that is representative of current through the electrodes 151, 152, FIG. 1, continuously or periodically. The ADC 250 can sample at a selected sampling rate.

At step 930, a measurement phase is selected. The measurement phase can be a selected phase (e.g., the first phase) in a phase sequence as described above.

At step 940, the resultant current signal (e.g., the output of the amplifier 193) is measured (e.g., by the ADC 250) at a plurality of measurement points, each corresponding to the selected measurement phase. This can be done as described above with reference to any of FIGS. 3-6B, e.g., using in-sequence undersampling, out-of-sequence undersampling, in-sequence phase shifting, or out-of-sequence phase shifting.

At decision step 950, it is determined whether there are more selected phases to measure. If so, the next step is step 930. If not, the next step is step 960. In this way, the selecting of step 930 and the measuring of step 940 are repeated so that a plurality of different measurement phases are selected. A respective plurality of points is measured for each of the plurality of different measurement phases.

At step 960, a phase difference corresponding to the fluid sample applied to the test strip is automatically determined, e.g., using the processor 186 of the hand-held test meter 100. This determination is based on the plurality of different measurement phases and the respective pluralities of measured data points. The phase difference can be determined using Fourier or other frequency-domain analysis, e.g., as described above with reference to FIGS. 7A-7C. In an example, the phase difference is determined using the frequency-domain magnitudes or phases of the fundamental frequency of the voltage signal 405 and the current signal 410, as measured at the various measurement phases. An example is discussed above with reference to the peaks 735, 740, FIG. 7C.

At optional step 970, a hematocrit value of the fluid sample is automatically determined by the processor 186. This determination is based on the determined phase difference using the processor. This step can be used, e.g., when the fluid sample is a whole blood sample. After step 970, blood glucose of the fluid sample can be determined, e.g., using the hematocrit value and an additional measurement of the fluid sample.

The processor 186 can compute the hematocrit by, for example, converting the measured data from the ADC 250 into a phase difference, as described above, and then employing a suitable algorithm or look-up table to convert the phase difference into a hematocrit value. Once apprised of the present disclosure, one skilled in the art will recognize that such an algorithm or look-up table can be configured to take into account various factors such as strip geometry (including electrode area and sample cell volume) and signal frequency.

It has been determined that a relationship exists between the reactance of a whole blood sample and the hematocrit of that sample. Electrical modeling of a bodily fluid sample (e.g., a whole blood sample) as parallel capacitive and resistive components indicates that when an alternating current (AC) signal is forced through the bodily fluid sample, the phase difference of the AC signal will be dependent on both the frequency of the AC voltage and the hematocrit of the sample. Moreover, modeling indicates that hematocrit has a relatively minor effect on the phase difference when the frequency of the signal is in the range of approximately 10 kHz to 25 kHz and a relatively significant effect on the phase difference when the frequency of the signal is in the range of approximately 250 kHz to 500 kHz. Therefore, the hematocrit of a bodily fluid sample can be measured by, for example, driving AC signals of known frequency through the bodily fluid sample and detecting their phase difference. For example, the processor 186 and the signal-measuring circuit 190 can be configured to measure the phase difference using the excitation voltage signal of a first frequency and a second excitation voltage signal of a second frequency. The phase difference of a signal with a frequency in the range of 10 kHz to 25 kHz can be used as a reference reading in such a hematocrit measurement, e.g., of a whole blood sample, while the phase difference of a signal with a frequency in the range of 250 kHz to 500 kHz can be used as the primary measurement. The phase difference of a signal with a frequency of about 75 kHz or higher, or of about 75 kHz to about 500 kHz, can also be used as the primary measurement.

### PARTS LIST FOR FIGS. 1-11

100 hand-held test meter
104 housing
106 strip port connector (SPC)
118 memory block
140 sample cell
150 analytical test strip
151, 152 electrodes
180 user interface buttons
181 display
186 processor
190 signal-measurement circuit
191 AC voltage source
192 resistor
193 amplifier
205 voltage source
210 switch
215 switch
220 calibration load
240 sample
245 low-pass filter
250 analog-to-digital converter (ADC)
290 square-wave generator
301,302 phases
310 current signal
311 measurement
314, 315, 316 points
320, 321 phases
330 curve
405 voltage signal
410 current signal
514 measurement
516 measurement
530 range marker
705 reference waveform
705 reference waveform
710 response waveform
725, 730 phases
735, 740, 782, 786, 790 peaks
900 method
910, 920, 930, 940 steps
950 decision step
960, 970 steps
1005 voltage supply
1015, 1017 switches
1020 dummy load
1040 fluid sample
1041, 1042 quadrature demodulators
1045, 1046 low-pass filters
1050 analytical test strip
1051, 1052 analog-to-digital converters (ADCs)
1086 processor
1093 transimpedance amplifier
1119, 1120, 1121 phases

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. References to “a particular embodiment” (or “aspect”) and the like refer to features that are present in at least one embodiment of the invention. Separate references to “an embodiment” or “particular embodiments” or the like do not necessarily refer to the same embodiment or embodiments; however, such embodiments are not mutually exclusive, unless so indicated or as are readily apparent to one of skill in the art. The word “or” is used in this disclosure in a non-exclusive sense, unless otherwise explicitly noted. It is intended that the following claims define the scope of the invention and that devices and methods within the scope of these claims and their equivalents be covered thereby.

What is claimed is:

1. A hand-held test meter for use with an associated analytical test strip, the hand-held test meter comprising:
a housing;
a square-wave generator disposed in the housing;
a two-stage transimpedance amplifier disposed in the housing;
a memory block storing a digital filtering algorithm; and
a processor disposed in the housing;
wherein the square-wave generator is configured to generate a square-wave voltage signal and to supply the generated square-wave voltage signal to an electrode of the analytical test strip inserted into the hand-held test meter;
the two-stage transimpedance amplifier is configured to receive from the analytical test strip a resulting current signal that originated from the square wave; and
the processor is configured to automatically execute the digital filtering algorithm to recover a fundamental phase and magnitude from the resulting current signal.
